SEQUENCE LISTING (4 sequences)

SEQ ID NO: 1 - DNA, 1549 bp, Homo sapiens
gaattcatgggtgcgagagcgtcaatattaagaggggaaaaattagataaatgggaaaagattaggttaaggccagggggaaagaaacattatatgttaaaacacatagtatgggcgagcagggagctggaaagatttgcacttaaccctggccttttagaaacatcagaaggatgtaaacaaataatgaaacagctacaaccagctctccagacaggaacagaggaacttaaatcattatacaacacagtagcaactctctattgtgtacatgaaaagatagaagtacgagacaccaaggaagccttagataagatagaggaagaacaaaacaaatgtcagcaaaaaacgcagcaggcaaaagcggctgacgggaaagtcagtcaaaattatcctatagtgcagaatctccaagggcaaatggtacatcaagccatatcacctagaaccttgaatgcatgggtaaaagtaatagaagaaaaggcttttagcccagaggtaatacccatgtttacagcattatcagaaggagccaccccacaagatttaaacaccatgttaaatacagtggggggacaccaagcagccatgcaaatgttaaaagatactattaatgaagaggctgcagaatgggatagattacatccagtccatgcggggcctattgcaccaggccagatgagagaaccaaggggaagtgacatagcaggaactactagtacccttcaggaacaaatagcatggatgacaagtaacccacctattccagtgggagacatctataaaagatggataattctggggttaaataaaatagtgagaatgtatagcccggtcagcattttggacataagacaagggccaaaggaaccctttcgagactatgtagatcggttctttaaaactttaagagctgaacaagctacacaagaagtaaaaaattggatgacagacaccttgttagtccaaaatgcgaacccagattgtaagaccattttgagagcattaggaccaggggctacattagaagaaatgatgacagcatgtcaaggggtgggaggacctggccacaaagcaagagtattggctgaggcaatgagtcaaacaaacagtggaaacataatgatgcagagaagcaattttaaaggccctagaagaattgttaaatgttttaactgtggcaaggaagggcacatagccagaaattgcagagcccctaggaaaaaaggctgttggaaatgtggaaaagaaggacaccaaatgaaagactgcactgagaggcaggctaattttttagggaaaatttggccttcccacaaggggaggccagggaatttccttcagaacagaccagagccaacagccccaccagcagagagcttcaggttcgaagagacaacccccgctccgaaacaggagccgatagaaagggaacccttaacttccctcaaatcactctttggcagcgaccccttgtctcaataaaagtagggggccagacaagggaggctctcttagacacaggagcagatgatacagtattgtcgac

SEQ ID NO: 2 - DNA, 1479 bp, Homo sapiens
atgggtgcgagagcgtcaatattaagaggggaaaaattagataaatgggaaaagattaggttaaggccagggggaaagaaacattatatgttaaaacacatagtatgggcgagcagggagctggaaagatttgcacttaaccctggccttttagaaacatcagaaggatgtaaacaaataatgaaacagctacaaccagctctccagacaggaacagaggaacttaaatcattatacaacacagtagcaactctctattgtgtacatgaaaagatagaagtacgagacaccaaggaagccttagataagatagaggaagaacaaaacaaatgtcagcaaaaaacgcagcaggcaaaagcggctgacgggaaagtcagtcaaaattatcctatagtgcagaatctccaagggcaaatggtacatcaagccatatcacctagaaccttgaatgcatgggtaaaagtaatagaagaaaaggcttttagcccagaggtaatacccatgtttacagcattatcagaaggagccaccccacaagatttaaacaccatgttaaatacagtggggggacaccaagcagccatgcaaatgttaaaagatactattaatgaagaggctgcagaatgggatagattacatccagtccatgcggggcctattgcaccaggccagatgagagaaccaaggggaagtgacatagcaggaactactagtacccttcaggaacaaatagcatggatgacaagtaacccacctattccagtgggagacatctataaaagatggataattctggggttaaataaaatagtgagaatgtatagcccggtcagcattttggacataagacaagggccaaaggaaccctttcgagactatgtagatcggttctttaaaactttaagagctgaacaagctacacaagaagtaaaaaattggatgacagacaccttgttagtccaaaatgcgaacccagattgtaagaccattttgagagcattaggaccaggggctacattagaagaaatgatgacagcatgtcaaggggtgggaggacctggccacaaagcaagagtattggctgaggcaatgagtcaaacaaacagtggaaacataatgatgcagagaagcaattttaaaggccctagaagaattgttaaatgttttaactgtggcaaggaagggcacatagccagaaattgcagagcccctaggaaaaaaggctgttggaaatgtggaaaagaaggacaccaaatgaaagactgcactgagaggcaggctaattttttagggaaaatttggccttcccacaaggggaggccagggaatttccttcagaacagaccagagccaacagccccaccagcagagagcttcaggttcgaagagacaacccccgctccgaaacaggagccgatagaaagggaacccttaacttccctcaaatcactctttggcagcgaccccttgtctcaataa

SEQ ID NO: 3 - protein, 513 aa, Homo aspiens
EFMGARASILRGEKLDKWEKIRLRPGGKKHYMLKHIVWASRELERFALNPGLLETSEGCKQIMKQLQPALQTGTEELKSLYNTVATLYCVHEKIEVRDTKEALDKIEEEQNKCQQKTQQAKAADGKVSQNYPIVQNLQGQMVHQAISPRTLNAWVKVIEEKAFSPEVIPMFTALSEGATPQDLNTMLNTVGGHQAAMQMLKDTINEEAAEWDRLHPVHAGPIAPGQMREPRGSDIAGTTSTLQEQIAWMTSNPPIPVGDIYKRWIILGLNKIVRMYSPVSILDIRQGPKEPFRDYVDRFFKTLRAEQATQEVKNWMTDTLLVQNANPDCKTILRALGPGATLEEMMTACQGVGGPGHKARVLAEAMSQTNSGNIMMQRSNFKGPRRIVKCFNCGKEGHIARNCRAPRKKGCWKCGKEGHQMKDCTERQANFLGKIWPSHKGRPGNFLQNRPEPTAPPAESFRFEETTPAPKQEPIEREPLTSLKSLFGSDPLSQKGARQGRLSTQEQMIQYCR

SEQ ID NO: 4 - protein, 492 aa, Homo sapiens
MGARASILRGEKLDKWEKIRLRPGGKKHYMLKHIVWASRELERFALNPGLLETSEGCKQIMKQLQPALQTGTEELKSLYNTVATLYCVHEKIEVRDTKEALDKIEEEQNKCQQKTQQAKAADGKVSQNYPIVQNLQGQMVHQAISPRTLNAWVKVIEEKAFSPEVIPMFTALSEGATPQDLNTMLNTVGGHQAAMQMLKDTINEEAAEWDRLHPVHAGPIAPGQMREPRGSDIAGTTSTLQEQIAWMTSNPPIPVGDIYKRWIILGLNKIVRMYSPVSILDIRQGPKEPFRDYVDRFFKTLRAEQATQEVKNWMTDTLLVQNANPDCKTILRALGPGATLEEMMTACQGVGGPGHKARVLAEAMSQTNSGNIMMQRSNFKGPRRIVKCFNCGKEGHIARNCRAPRKKGCWKCGKEGHQMKDCTERQANFLGKIWPSHKGRPGNFLQNRPEPTAPPAESFRFEETTPAPKQEPIEREPLTSLKSLFGSDPLSQ

(19) United States
(12) Patent Application Publication
(10) Pub. No.: US 2006/0210585 A1
(43) Pub. Date: Sep. 21, 2006

(54) Method for the production of hiv-1 gag virus-like particles

(76) Inventors: Ann Jaffray, Plumstead (ZA); Anna-Lise Wiliamson, Pinelands (ZA); Edward Peter Rybicki, Pinelands (ZA)

Correspondence Address: JOHN S. PRATT, ESQ;KILPATRICK STOCKTON, LLP, 1100 PEACHTREE STREET, ATLANTA, GA, 30309, US

(21) Appl. No.: 10/537,557

(22) PCT Filed: Dec. 4, 2003
(86) PCT No.: PCT/IB03/05634

§ 371 Date: Oct. 7, 2005

(30) Foreign Application Priority Data
Dec. 4, 2002 (ZA) 2002/9830

(51) Int. Cl. A61K 39/12; C12Q 1/68

(52) U.S. Cl. 424/199.1; 424/204.1; 435/6

(57) ABSTRACT

The invention describes a vector including a nucleotide sequence encoding an HIV Gag polypeptide for use in the production of HIV-1 Gag virus-like particles. The vector may be a plant vector, for example, a tobacco mosaic virus vector such as the pBSG1057 vector or a tobacco etch virus vector. The vector may also be an Agrobacterium tumefaciens containing a T-derived plasmid construct. Alternatively, the vector may be an insect vector such as a baculovirus vector. HIV-1 Gag virus-like particles are also described, as is the use of the virus-like particles in a vaccine for use in the treatment or prophylaxis of HIV infection in a mammal, the vaccine including virus-like particles of proteins or polypeptides substantially as described above.

### FIELD OF THE INVENTION

This invention relates to a method for the production of HIV-1 Gag virus-like particles, to the virus-like particles prepared by the method, and to the use of the virus-like particles in a vaccine.

### BACKGROUND OF THE INVENTION

The HIV genome contains three main open reading frames. The gag open reading frame (FIG. 1) encodes a 55 kDa precursor protein (Pr55^{Gag}) which is cleaved further by an HIV-encoded protease during virion maturation into three major structural proteins, a regulatory domain and 2 spacer peptides (Luciw, 1996). The structural proteins include the matrix (MA) protein (P17-AA1 to AA132), the capsid (CA) protein (P24-AA133 to AA363) and the nucleocapsid (NC) protein (P9-M377 to AA432). The regulatory domain (P6) spans AA449 to AA500 while the spacer regions P1 and P2 are located from AA433 to AA448 and AA364 to AA376 respectively (von Schwedler et al., 1998).

The pol open reading frame overlaps that of gag from AA430 and is expressed via a ribosomal frame-shifting event that occurs at a frequency of 5 to 10% during translation to produce a Gag-Pol precursor protein of 160 kDa (Pr160) (Jacks et al., 1988). The pol gene encodes several open reading frames including that for the protease, reverse transcriptase, RNase H and integrase enzymes of HIV-1. The env open reading frame lies further downstream of pol and encodes a 160 kDa precursor protein (gp160) of the viral envelope proteins gp41 and gp120 (Luciw, 1996).

After infection of a host cell, HIV-1 RNA is reverse transcribed into DNA which is subsequently integrated into the host genome (proviral stage). The Gag and Gag-Pol precursors are translated from transcribed HIV-1 provirus RNA in the cytosol and targeted to the host cell membrane. The Gag precursor associates with two copies of viral RNA and interacts with the Gag-Pol precursor to assemble into particle-like structures which line the host-cell membrane. They aggregate in such a way as to induce membrane curvature and subsequent bud formation during which viral Env proteins are also incorporated into the forming particles. The particles pinch off the membrane after which the HIV-1 particle maturation occurs, with the protease cleaving Gag and Gag-Pol into mature structural and functional proteins which lead to core condensation and thus a mature infectious virion.

Pr55 Gag has been shown to assemble into virus-like particles (VLPs) in the absence of any other HIV-encoded genes in both mammalian and insect cells. These particles closely resemble the morphology of immature HIV virions and are non-infectious (Overton et al., 1989; Gheysen et al., 1989; Royer et al., 1991; Royer et al., 1992; Shioda and Shibuta, 1990; Vernon et al., 1991; Mergener et al., 1992). A number of Gag domains have been shown to be important in driving this particle assembly process and it has been shown that in fact about 80% of this precursor protein can be either deleted or replaced by heterologous sequences without significantly compromising VLP production (Accola et al., 2000). These important domains are discussed below with respect to the functions of the individual proteins comprising Gag.

MA Protein (p17)

The MA domain of Gag (FIG. 2) comprises a total of 132 amino acids and is responsible for targeting Gag precursor protein to the plasma membrane and virus-like particle assembly. The M domain (retrovirus membrane-binding domain) at the N-terminal of MA is mostly responsible for this function. MA has an N-terminal glycine residue which has been shown to be required for targeting Gag to the host cell membrane and facilitating particle assembly (Gheysen et al., 1989). For this to occur, the glycine residue has to be myristylated. The amino acid recognition sequence for myristylation to occur at the N-terminus of Gag is gly-x-x-x-ser/thr.

The targeting and accumulation of HIV-1 Gag precursor at the host cell membrane by myristylation has been shown to occur in baculovirus-infected yeast cells, insect cells and mammalian cells (Jacobs et al., 1989; Gheysen et al, 1989; Bryant and Ratner, 1990). Substitution of the glycine residue erradicated particle formation, complementation of the residue restored VLP production and when using myr mutants, Gag precursor was shown to accumulate in infected cell cytoplasm but did not associate with the host cell membrane. The myristyl moiety is thus required for stable membrane association of the particles. Only complete inhibition of Gag myristylation prevents VLP budding (Morikawa et al., 1996), i.e. only a few myristylated Gag molecules are sufficient for plasma membrane targeting and budding.

Spearman (1997) has shown that myristate is the primary determinant of Gag-host cell membrane stability and therefore critical for particle assembly. Paillart and Göttlinger (1999) have proposed a model from results showing that the N-terminus of Gag is critical for insertion into the host cell membrane.

Apart from the myristylation signal other regions of MA have been shown to be important for targeting Gag to the host cell membrane and subsequent particle assembly. Fäcke et al., (1993) showed that a large deletion of MA (AA16 to 99) caused drastic alteration of particle morphogenesis leading to immature particles produced in the endoplasmic reticulum instead. MA is required for the proper assembly of envelope proteins into the virion.

Yuan et al. (1993) showed that various MA deletions and substitutions caused a dramatic reduction in virus particle production. They demonstrated that it is possible that a polybasic region in MA (AA20 to 32) serves as part of a Gag transport signal to the membrane.

Zhou et al., (1994) studied this polybasic region further and showed that the highly basic residues form a positively charged surface which interacts with negatively charged phospholipids on the inner face of the lipid bilayer of the plasma. Ono and Freed (1999) have shown that a single mutation of MA (AA6 from V to R) severely impaired membrane binding without affecting myristylation.

Capsid (CA) Protein (p24)

The CA domain (FIG. 3) encodes a protein of approximately 230 amino acids in length and has several domains which appear to be important for particle assembly, the first of which is a major homology region (MHR). The region extending from the N-terminus of CA downstream to the MHR is dispensible for particle formation, but any further deletions extending further into the MHR impair particle production (Borsetti et al., 1998). Zhao et al., (1994) also showed that baculovirus constructs of HIV-1 CA with a 10-amino acid deletion of AA140-150 as well as a separate deletion of AA250-260 led to the accumulation of viral protein at the cell membrane of insect cells. However there was no particle assembly or extracellular budding indicating that these two regions of CA at least, must play some role in normal particle formation.

On the other hand, Borsetti et al. (1998) showed that efficient particle formation occurred in the absence of both MA (excluding the myristyl anchor) and the N-terminal of the CA domain and therefore concluded that there is no distinct region between the myristyl anchor and MHR which is absolutely essential for efficient particle release or assembly. They concluded that the C-terminal half of Gag contains protein-protein interaction domains which are essential for efficient particle assembly.

It seems that the C-terminal sequences may be required for protein-protein interactions but are not required for spherical particle formation and that the sphere is determined by the presence of an N-terminal extension on the CA domain.

The presence of RNA (heterogeneous in size and of viral and cellular origins) within the particles has been reported (Gheysen and Shioda and Shibuta)

Truncations into the P2 spacer regions have been shown to abolish particle formation.

Spacer Region 2 (p2)

Borsetti et al., 1998 have shown that the presence or absence of p2 determines the assembly of Gag proteins into spherical particles or cylindrical particles respectively. Morikawa et al. (2000) have also verified that this region is essential for VLP production in that if this region is truncated in any way, VLP production is abolished.

Nucleocapsid (NC) Protein (p7)

The NC domain (FIG. 4) has been shown to contain two well-conserved Cys-His boxes resembling zinc finger motifs often found in DNA binding proteins. These are thought to play a role in RNA binding and encapsidation but influence some other aspects of particle assembly as well. There are two highly basic regions flanking these two motifs which have been shown to influence RNA binding in vitro and RNA encapsidation into virions if mutated. Jowett et al. (1992) showed that the deletion of the second Cys-His box did not affect particle formation but reduced RNA binding substantially. However, they also showed that deletion of both Cys-His boxes encouraged the formation of larger particles and the loss of RNA binding altogether. The deletion of sequences upstream of the Cys-His boxes caused the abolition of particle-forming ability.

Dawson and Yu (1998) showed that the NC domain is essential for efficient assembly of HIV-1 and for the production of particles with wildtype density.

In addition to the Cys-His boxes, an I domain (interaction or assembly domain) close to the N-terminal of NC has been identified which is responsible for the formation of Gag protein complexes and also for the formation of punctate foci of Gag proteins at the plasma membrane. There are two positively charged basic arginine residues (AA380 and AA384) which have been shown to be critical for the function of the N-terminal I domain (interaction or assembly domain). Sandefur et al., (2000) have shown that I domain-deficient mutants block the formation of budding VLPs.

Spacer Region 1 (P1)

CA and NC are separated by a short spacer region SP1 which is a protease cleavage site. Wiegers et al. (1998) have shown that when cleavage of SP1 from NC is prevented, maturation of particles is delayed and the ribonucleoprotein core has an irregular morphology. However, when SP1 cleavage from CA is prevented, normal condensation of the ribonucleoprotein cone occurs but capsid condensation is prevented. They concluded from this that HIV maturation is a sequential process controlled by the rate of cleavage at individual sites.

P6 Protein

Elements important for controlling particle size are contained within the C-terminal region of gag (P6) (FIG. 5) as various deletions and substitutions of this region have been shown to induce the formation of very large particles (Garnier et al., 1998). A specific domain referred to as the late (L) domain has been identified in P6 that is critical for the virus-cell separation step. This region contains a PTAPP amino acid sequence. Sequences downstream of this domain in P6 were shown to be dispensable for virus release.

Other Features of Gag Influencing VLP Formation

Buck et al. (2001) have recently found that mRNA of the HIV-1 Gag open reading frame exhibits internal ribosome entry site (IRES) activity that promotes translational initiation of Gag, producing a 40 kDa Gag protein. This IRES is located at an internal AUG codon found near the N-terminus of CA. This may have consequences when Gag is expressed in in vitro systems since initiation of translation is thought to be promoted by the direct binding of ribosomes with the participation of other host cell factors quite independently of the mRNA cap.

Minimal HIV-1 Gag Sequences Required for VLP Assembly and Release

Although numerous domains of Gag have been shown to perform a particular function in Gag VLP assembly and release, deletion mutants of Gag have indicated that a number of these can be dispensable, and assembly of VLPs has been shown to be surprisingly tolerant to significant Modifications of Gag protein (Wang et al., 1998; Accola et al., 2000; Wilk et al., 2001). Wang et al., (1998) made a series of C-terminal truncated mutants with which to examine VLP-producing capabilities. Truncated Gag precursors lacking most of the C-terminal Gag assembled into particles and were released from mammalian cells. A mutant with most of MA and the entire p6 region deleted still produced particles although less than wildtype particles. The smallest Gag product capable of VLP assembly was a 28 kDa protein which consisted of a few MA amino acids and the CA-p2 domain. The N-terminal portions of CA appeared to be critical when most of the MA domain was deleted, suggesting a requirement for an intact CA domain to assemble and release particles. Accola et al., (2000) showed that 80% of Gag could be deleted or replaced by heterologous sequences without significantly compromising VLP production. The smallest chimeric molecule still able to efficiently form VLPs was 16 kDa. This construct contained a leucine zipper domain of the yeast transcription factor GCN4 to substitute for the assembly function of nucleocapsid, followed by a PPPPY motif to provide the L domain function, and retained only the myristylation signal and the C-terminal CA-p2 domain of Gag.

Ability of Gag to Stimulate an Immune Response

The long-term solution to combating the AIDS epidemic is through immunisation with a suitable vaccine and there are numerous HIV-1 genes and epitopes which are currently being used to develop vaccines. Although there is no Gag vaccine in use to date, a number of studies have shown that this protein induces an immune response in HIV-1-infected patients as well as in animals challenged with novel vaccines (Friedman et al., 2000; Revskaya and Frankel, 2001).;). This response has been shown in some cases to be both humoral and cell-mediated (Qui et al., 1999; Leung et al., 2000; Qui et al., 2000; Montefiori et al., 2001; Kazanji et al., 2001).

Goulder et al. (2000) did studies to identify the epitopes, which dominate the CTL response in ethnic groups and age groups worst hit by the global epidemic. They focussed on Gag because a number of gag specific responses have been shown to be associated with protection in HIV infection (Nixon and McMichael, 1991; Riviere et al., 1995). The immunodominant Gag-specific CTL responses appeared to be focussed on 3 highly immunogenic regions, which together spanned 16% of the total length of Gag p17 and Gag p24 proteins but which represent two thirds of the dominant Gag-specific CTL responses detected. These results suggest that Gag would be an ideal candidate for vaccine design.

Plants as Sources of Vaccines

There are many examples of the use of plants as sources of foreign protein and they are considered as viable and competitive expression systems for large-scale protein production (Doran, 2000). Favourable reasons for their use include the potential for large-scale, low-cost biomass production, a low risk of contamination by mammalian viruses and other animal pathogens, the ability of plant cells to correctly fold and assemble multimeric proteins, and a low processing requirement for proteins administered orally in plant food or feed. They are thus considered a viable option for the production of foreign proteins, which can be used as vaccines.

A number of potentially useful vaccine candidates have been produced in plants and tested in animals. A useful technique for introducing foreign genes into plants has been via viral vector transmission.

Usha et al., (1993) have used cowpea mosaic virus (CPMV) particles to express epitopes of foot and mouth disease virus (FMDV) on their coat protein as a result of a fusion on the coat protein gene.

Koo et al. (1999) made hybrids of tobacco mosaic virus (TMV) by fusing short epitopes from murine hepatitis virus (MHV) to TMV coat protein and subsequently propagating them in tobacco plants.

Fernández-Fernández et al., (2001) have developed a plum pox potyvirus vector for the expression of foreign proteins. They have used it to express an antigenic structural protein of rabbit hemorrhagic disease virus (RHDV), producing chimeric virus particles which when inoculated into rabbits produce an immune resporise against RHDV.

A number of other methods have been used to produce vaccine candidates in plants. McCormick et al. (1999) demonstrate the modification of a TMV vector such that it not only produces single chain Fv fragments in plants, but secretes them into the apoplast. This makes harvesting of the product a lot simpler than having to isolate foreign proteins from leaf extracts. Several attempts have also been made to make plants transgenic for production of foreign proteins to be used as vaccine candidates. Mason et al. (1996) have made transgenic tobacco and potato plants to successfully produce Norwalk virus capsid protein, which shows immunogenicity in mice. Wigdorowitz et, al. (1999) have made transgenic alfalfa plants expressing an antigenic protein against FMDV and shown that animals immunised with purified antigen show immunogenicity against the virus.

There have been a few attempts at producing candidate vaccines against HIV-1 in plants. Yusibov et al. (1997) have used the coat protein of alfalfa mosaic virus as a carrier molecule to express antigenic peptides from HIV-1 (V3 loop). In vitro transcripts of recombinant virus with sequences encoding the antigenic peptides were synthesised from DNA constructs and used to inoculate tobacco plants. Recombinant virus particles were produced and purified and used for immunisation of mice. The antigens elicited specific virus-neutralizing antibodies in immunised mice. Zhang et al., (2000) used a tomato bushy stunt virus (TBSV) as an expression vector of HIV-1 p24 protein. This gene was introduced into the TBSV genome as an in-frame fusion with a 5′ terminal portion of the TBSV coat protein ORF. Introduction into plants led to the accumulation of p24 fusion proteins in inoculated leaves.

There has, to date, been no report in the literature about the ability of GAG VLPs to assemble correctly in plants. Although some proteins, and even some HIV proteins, have been shown to assemble into VLPs in plants and insects, many other proteins do not assemble at all, or do not assemble correctly, in plants or insects, and mammalian studies are not reliable indicators of whether VLPs will similarly be produced in plants or insects.

### SUMMARY OF THE INVENTION

According to a first embodiment of the invention, there is provided a vector including a nucleotide sequence encoding an HIV Gag polypeptide, wherein the nucleotide sequence encoding the Gag polypeptide comprises a sequence having at least 90% sequence identity to the sequence set forth in SEQ ID NO: 1.

According to a second embodiment of the invention, there is provided a vector including a nucleotide sequence encoding an HIV Gag polypeptide, wherein the nucleotide sequence encoding the Gag polypeptide comprises a sequence having at least 90% homology to the sequence set forth in SEQ ID NO: 2.

The vector of either embodiment may be a plant virus vector, for example, a tobacco mosaic virus-derived cDNA cloned vector such as the pBSG1057 vector, or a potyvirus-derived cDNA such as tobacco etch virus (TEV) or turnip mosaic virus (TuMV). The vector may also be an Agrobacterium tumefaciens containing a T-derived plasmid construct.

Alternatively, the vector may be a baculovirus vector, such as the bacmid vector.

According to a third embodiment of the invention, there is provided a cell including a vector substantially as described above, wherein the nucleotide sequence is operably linked to control elements compatible with expression in the cell.

The cell may be a plant or insect cell. For example, the cell may be an N. benthamiana plant cell or an Sf 21, Sf 9 or the like cell.

According to a fourth embodiment of the invention, there is provided a method of producing an HIV-1 immunogenic protein or a related polypeptide, the method comprising the steps of:

introducing a vector or vector system into a host cell, the vector or vector system including a nucleic acid sequence encoding the HIV-1 immunogenic protein or related polypeptide derived by substitution, deletion and/or insertion of one or more nucleotides, and/or extension or truncation of one or both ends thereof, the nucleic acid sequence having at least 90% identity to the sequence set forth in SEQ ID NO:1;

causing expression of the nucleic acid sequence in the host cell; and

recovering the resulting HIV-1 immunogenic protein or related polypeptide produced within the host cell.

According to a fifth embodiment of the invention, there is provided a method of producing an HIV-1 immunogenic protein or a related polypeptide, the method comprising the steps of:

introducing a vector or vector system into a host cell, the vector or vector system including a nucleic acid sequence encoding the HIV-1 immunogenic protein or related polypeptide derived by substitution, deletion and/or insertion of one or more nucleotides, and/or extension or truncation of one or both ends thereof, the nucleic acid sequence having at least 90% identity to the sequence set forth in SEQ ID NO:2;

causing expression of the nucleic acid sequence in the host cell; and

recovering the resulting HIV-1 immunogenic protein or related polypeptide produced within the host cell.

The vector and host cell may be substantially as described above.

According to a further embodiment of the invention, there is provided an HIV-1 protein or polypeptide that is produced according to the method substantially as described above.

The protein may be an HIV-1 Pr55 Gag protein, and may be assembled into the form of virus-like particles (VLPs).

According to a further embodiment of the invention, there is provided a vaccine for use in the treatment or prophylaxis of HIV infection in a mammal, the vaccine including virus-like particles of proteins or polypeptides substantially as described above.

The vaccine may induce an immunogenic response to the virus-like particles in a suitable susceptible host.

The vaccine may include a pharmaceutical excipient and/or adjuvant, and a therapeutically effective amount of the virus-like particles.

### DESCRIPTION OF THE DRAWINGS

FIG. 1 shows a schematic representation of the gag open reading frame of the HIV-1 genome;

FIG. 2 shows a schematic representation of the matrix (MA) protein (p24) domain of the gag gene of FIG. 1;

FIG. 3 shows a schematic representation of the capsid (CA) protein (p24) domain of the gag gene of FIG. 1;

FIG. 4 shows a schematic representation of the nucleocapsid (NC) protein (p7) domain of the gag gene of FIG. 1;

FIG. 5 shows a schematic representation of the domain of the p6 protein domain of the gag gene of FIG. 1;

FIG. 6 shows the DNA sequence of the Du422 gag sequence used for cloning into pBSG1057 (SEQ ID NO: 1). The bold underlining represents the gag gene and the dotted underlining represents the partial pol fragment;

FIG. 7 shows a plasmid map of pBSG1057;

FIG. 8 shows a plasmid map of pBSGgag6;

FIG. 9 shows a plasmid map of pBSGgagopt11;

FIG. 10 shows the DNA sequence of the native Du422 gag sequence (SEQ ID NO: 2);

FIG. 11 shows HIV-1 subtype C Pr55 Gag VLPs resulting from gagopt expression immunotrapped onto carbon-coated grids using anti-p17 monoclonal antibody (Chemicon) (the bar represents 100 nm);

FIG. 12 shows (a) HIV-1 subtype C Gag VLPs produced in transfected Sf21 cells and (b) Gag VLPs budding into the extracellular medium from Sf21 plasma membrane (the bar represents 100 nm);

FIG. 13 shows the amino acid sequence of the nucleotide sequence of FIG. 6 (SEQ ID NO: 3); and

FIG. 14 shows the amino acid sequence of the nucleotide sequence of FIG. 10 (SEQ ID NO: 4).

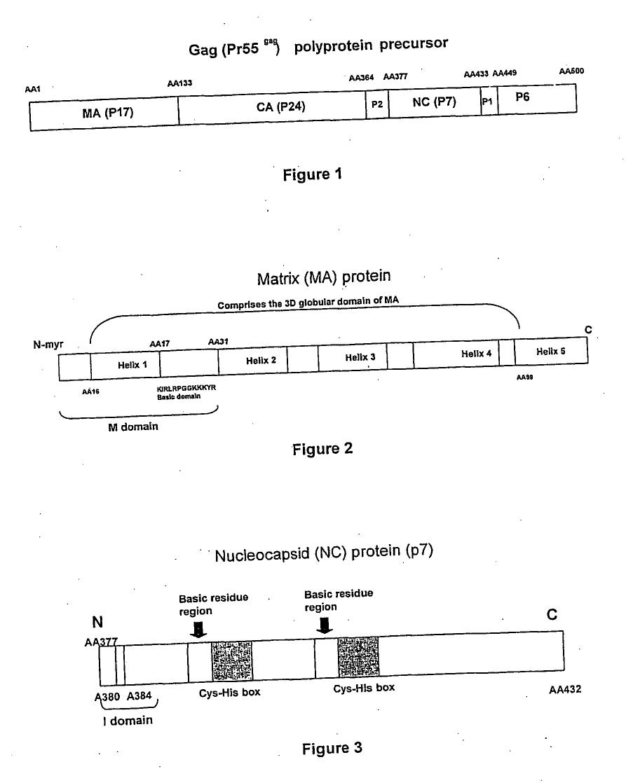

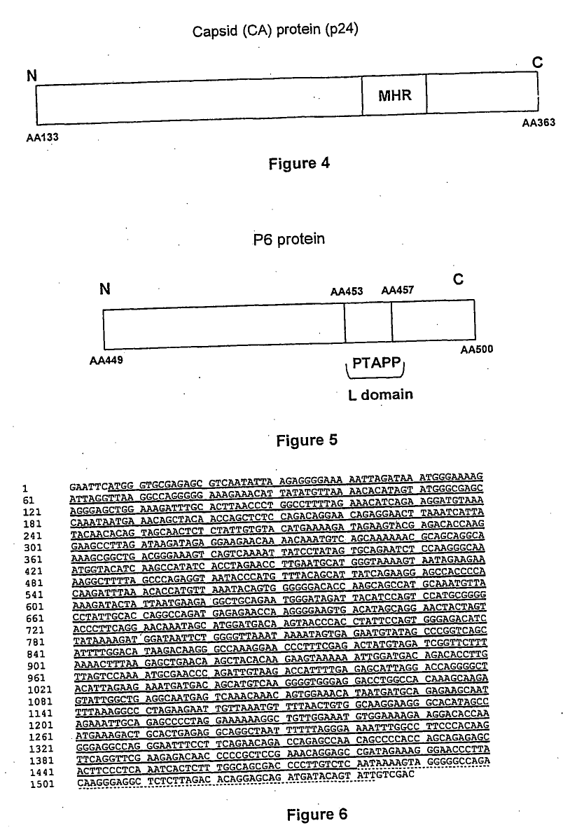

### DETAILED DESCRIPTION OF AN EMBODIMENT OF THE INVENTION

The invention will now be described in more detail with reference to particular embodiments of the invention.

Cloning of Gag into TMV Vector pBSG1057

Transient expression in tobacco was achieved using the vector plasmid pBSG1057 from Large Scale Biology Corporation. When in vitro transcribed into RNA, this vector provides an infectious engineered tobacco mosaic virus which expresses native or plant codon optimised Du422 Pr 55 Gag.

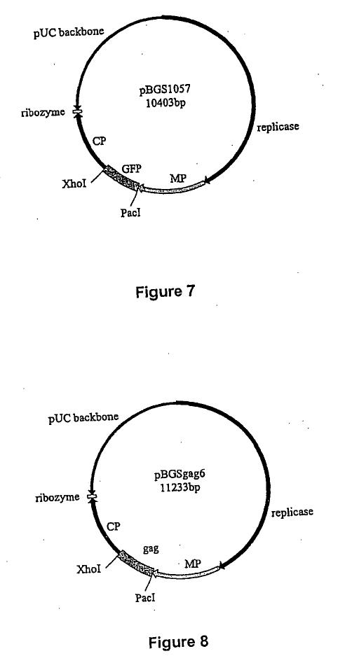

The Gag gene was obtained from HIV-1 isolate DU422 (obtained from a South African sex worker cohort, and assigned provisional accession no. 01032114 by the European Collection of Cell Cultures) (FIG. 6). It comprises the entire gag gene sequence and the first 57 bases of the pol gene sequence (SEQ ID NO: 2). It was cloned into the EcoRI and SalI restriction enzyme sites of an E. coli vector pGEM-T easy™. The ends of the gene were modified by PCR amplification such that PacI and XhoI restriction enzyme sites were attached to the 5′ and 3′ ends respectively, to facilitate cloning into the TMV vector pBSG1057 (FIG. 7). Amplification products were re-cloned into pGEM-T easy™ and sequenced to verify the integrity of the restriction enzyme sites and the gag sequence.

The green fluorescent protein (GFP) gene sequence was excised from pBSG1057 by restriction enzyme digestion with PacI and XhoI, and gag cloned into the TMV vector at these 2 sites to produce the clone pBSGgag6 (FIG. 8).

It is theorised that the identical gag construct could be cloned in frame into a potyvirus-derived cDNA clone, flanked by appropriate endoproteinase recognition sequences derived from a potyvirus proteome, for expression via in vitro-transcribed infectious recombinant potyviral RNA. The same would be true for vectors derived from any plant virus.

It is also theorized that large-scale transient expression in tobacco could be performed by means of infiltrating tobacco leaves with an Agrobacterium suspension. Using this method, many cells could be transformed and the protein could be produced somatically rather than first having to produce callus and then transgenic plants (Kapila et al., 1997). The vector may be Agrobacterium tumefaciens containing a T-derived plasmid construct.

A tobacco etch virus, such as those available from Large Scale Biology Corporation, could also be used in the invention.

Codon-Optimisation of Gag Gene for Nicotiana

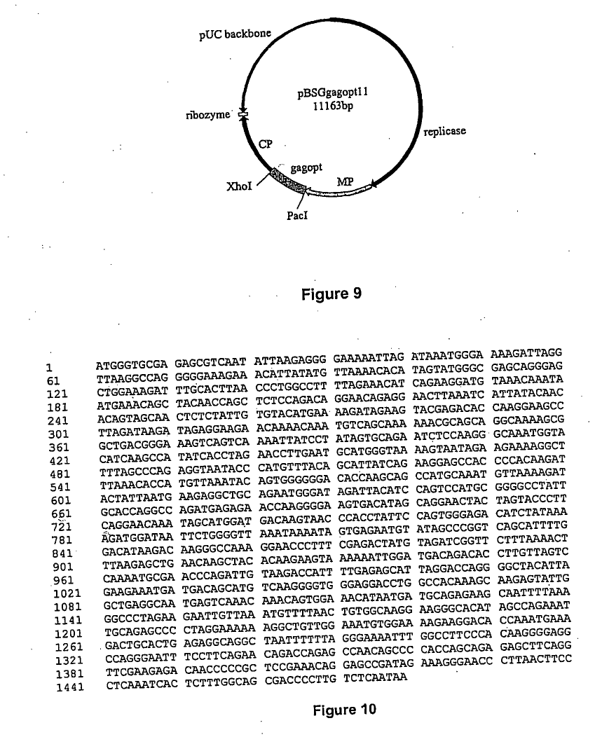

An additional strategy was undertaken to codon-optimise the gag gene for Nicotiana, synthesise it, and clone it using a similar strategy to that described above Into the pBSG1057 TMV vector with the hope that this gene would enhance Gag protein expression when introduced into N. benthamiana. The codon-optimised gag gene sequence (SEQ 10 NO: 1) was cloned into the PacI and XhoI restriction enzyme sites of pBSG1057 after removal of GFP. The resultant clone was called pBSGgagopt11 (FIG. 9).

Transcription of pBSGgag6, pBSGgagopt11 and pBSG1057

mRNA of pBSGgag6, pBSGgagopt11 and pBSG1057 was produced using a Ribomax Transcription/translation kit (Promega). Ten micrograms of each plasmid was used per reaction.

Infection of Nicotiana Benthamiana with Recombinant TMV mRNA

N. benthamiana plants were inoculated with mRNA transcripts of the pBSGgag6 and pBSGgagopt11 clones as well as with the TMV vector containing GFP (pBSG1057) as described previously. Water-inoculated plants served as negative controls.

The mRNA (50 μl) was rubbed over an expanding leaf of 6-week old N. benthamiana plants using cotton-wool buds. Plants were grown under normal growth conditions in plant rooms and monitored daily with a UV light for the appearance of green fluorescent spots (GFP) in both inoculated and upper leaves of the control plants inoculated with pBSG1057 mRNA transcripts, as well as for TMV symptoms in pBSGgag6-, pBSGgagopt11- and pBSG1057-inoculated plants. Systemic spread of GFP was used as an indicator of systemic spread of recombinant TMV and leaves were sampled for detection of Gag protein by western blotting, EM analysis and ELISA.

TMV Symptoms and GFP Fluorescence:

Green fluorescent spots were visible under the UV light on the inoculated leaves of those infected with pBSG1057 mRNA transcript at 4 days post inoculation (dpi). Spread of the GFP spots to upper leaves was visible at 10 dpi. TMV symptoms were visible in the newer growth of pBSG1057-inoculated plants at 17 dpi and in the pBSGgag6- and pBSGgagopt11-inoculated plants at 24 dpi.

Detection of Gag Protein

Crude protein preparations were made by crushing up leaves using a mortar and pestle, filtration of remaining solid matter through cheesecloth, and addition of loading buffer.

Western Blotting

The samples were boiled for 5 minutes and run on 10% SDS polyacrylamide gels to separate the proteins. Resolved proteins were transblotted from the SDS polyacrylamide gels onto nitrocellulose membranes. Membranes were probed with an anti-mouse p17 monoclonal antibody (Chemicon) at a dilution of 1:200.

Lanes containing crude protein preparations from pBSGgag6- and pBSGgagopt11-inoculated leaves did not yield any positive result compared with baculovirus-produced gag (see below) which highlighted the presence of a 55 kD protein.

EM Analysis for Gag VLPs (Immunotrapping)

Crude apical leaf extracts (35 dpi) were ground up in PBS (pH 7.4) and centrifuged at low speed to pellet remaining unground tissue. Small amounts of leaf extract were dried on copper grids and immunotrapped with an anti-mouse p17 monoclonal antibody (Chemicon) at a dilution of 1:200. The grids were counterstained with uranyl acetate and viewed using a transmission electron microscope.

There was very little plant material present in the pBSG1057-inoculated sample (no particles ranging from 100 to 120 nm in size) and very few TMV particles.

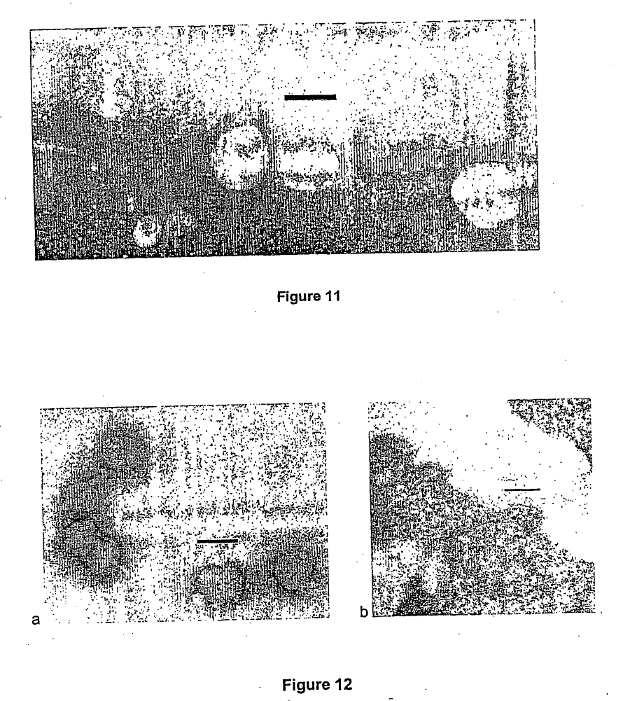

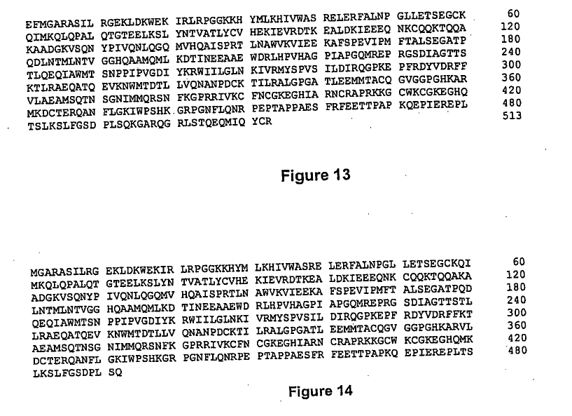

Apparent virus-like particles which were morphologically identical to baculovirus-expressed Gag Pr55 VLPs (FIG. 12) were detected in concentrated extracts made from N. benthamiana plants (FIG. 11). In the pBSGgag6-inoculated sample there were a large number of 100 to 110 nm-sized VLPs as well as quite a few TMV subunit-sized particles (25 nm). In the pBSGgagopt11-inoculated sample, there were similar shaped but less 100 to 110 nm-sized particles to those seen in the pBSGgag6-inoculated sample.

ELISA

Crude leaf extracts were centrifuged at low speed to pellet remaining unground tissue. Small amounts were diluted in 1×PBS and used for detection of p24 antigen by using the Vironostika® p24 HIV-1 antigen ELISA kit.

Preliminary results of ELISA performed on crude preparations of protein showed the presence of gag protein in pBSGgagopt11-inoculated leaves at a level of 460 pg/ml crude preparation protein solution. Expression of the gagopt gene in N. benthamiana plants systemically infected with the appropriate vectors was significantly higher than native gag gene.

Baculovirus Expression of HIV-1 Subtype C Gag VLPs

HIV-1 subtype C Gag VLPs were produced using the Bac-to-Bac® baculovirus expression system (Life Technologies). These provide a relevant positive control for further protein (Gag) detection experiments and can be used to generate antibodies specific to HIV-1 subtype C Gag.

The HIV-1 subtype C gag gene from the South African HIV isolate Du422 (Williamson et al., 2003) (SEQ ID NO: 2) was cloned into the multiple cloning site pFastBac1, and transposed into competent E. coli DH10Bac cells which were then screened for successful transposition into the baculovirus shuttle vector (bacmid).

Gag VLPs were produced in Spodoptera frugiperda (Sf21) cells via recombinant baculovirus expressing the full-length myristylated Pr55Gag precursor protein, according to the manufacturer's protocols (Gibco Life Sciences). The cells were incubated in TC100 medium (Gibco Life Sciences) supplemented with foetal calf serum at 28° C. for 84 h.

Because the cloned gag gene contains an N-terminal glycine residue sequence, it is expected that the recombinant protein be targeted to the host plasma membrane. VLPs thus formed were subsequently budded from the cell surface into the insect cell medium. Transfected Sf21 cells were separated from VLPs which had budded into the culture medium by centrifugation at 3000 g. Putative Pr55Gag VLPs were purified from the culture fluid on sucrose gradients as described by Nermut et al. (1994). Purified VLPs were dialysed for 16 h in 1× phosphate-buffered saline (PBS) at 4° C. and Gag content and integrity was evaluated by western blotting using antiserum to HIV-1 p17 (ARP431, NIBSC) diluted 1 in 1000 in 1×PBS (pH 7.4) after SDS-PAGE on 10% gels.

The process of VLP production by Sf21 cells was visualised by transmission electron microscopy (TEM). Recombinant virus-infected cells were prepared for ultrathin sectioning by fixing cells sequentially in 2.5% glutaraldehyde and 1% osmium tetroxide in 1×PBS (pH 7.4). Fixed cells were washed in 1×PBS and water, and then dehydrated in graded ethanol solutions and 100% acetone, after which they were embedded in Spurrs resin and sectioned. Sections were stained with both 2% uranyl acetate and Reynolds lead citrate and viewed using a Zeiss S1109 electron microscope at magnifications of 12000× to 100 000′ using an accelerating voltage of 80 kV.

Gag VLPs harvested from the extracellular medium were prepared for TEM by adsorption onto carbon-coated copper grids and staining with 2% uranyl acetate or 2% methylamine tungstate (FIG. 12).

VLPs of approximately 110 to 120 nm in diameter were Visualized under the electron microscope, verifying successful gag VLP production. A single 55 kD protein band was visualized in samples resolved on an SDS page gel, and a monoclonal and a polycolonal antibody to gag P17 protein were found to react positively using western blot analysis. Two additional monoclonal antibodies to gag P24 protein were tested subsequently against baculovirus-derived VLPs and reacted positively using western blot analysis.

Vaccine Development

It is envisaged that the immunogenic VLPs produced in the plant and insect cells, as described above, will be used in the manufacture of a vaccine for use in the treatment or prophylaxis of HIV infection in a mammal. The vaccine would be expected to induce an immunogenic response to the virus-like particles in the mammal. In addition to a therapeutically effective amount of the virus-like particles, the vaccine could include a pharmaceutical excipient and/or adjuvant.

Although the invention has been described above with reference to particular embodiments, it is not intended that this should limit the invention to what has been described above.

### REFERENCES

Accola, M. A., Strack, B. and Göttlinger, H. G. 2000. Efficient particle production by minimal Gag constructs which retain the carboxy-terminal domain of human Immunodeficiency virus type 1 capsid-p2 and a late assembly domain. J. Virol. 74: 5395-5402.
Borsetti, A., Öhagen, Á. and Göttlinger, H. G. 1998. The C-terminal half of the human Immunodeficiency virus type 1 gag precursor is sufficient for efficient particle assembly. J. Virol. 72: 9313-9317.
Buck, C. B., Shen, X., Egan, M. A., Pierson, T. C., Walker, C. M. and Siliciano, R. F. 2001. The human immunodeficiency virus type 1 gag encodes an internal ribosome entry site. J. Virol. 75: 181-191.
Bryant, M. and Ratner, L. 1990. Myristolation-dependent replication and assembly of human immunodeficiency virus 1. Proc. Natl. Acad. Sci. USA 87: 523-527.
Dalsgaard, K., Uttenthal, Á., Jones, T. D., Xu, F., Merryweather, An., Hamilton, W. D. O., Langeveld J. P. M., Boshuizen, R. S., Kamstrup, S., Lomonossoff, G. P., Porta, C., Vela, C., Casal, J. I., Meloen, R. H. and Rodgers P. B. 1997. Plant-derived vaccine protects target animals against a viral disease. Nature Biotech. 15: 248-252.
Dawson, L. and Yu, X-F. 1998. The role of the nucleocapsid of HIV-1 in virus assembly. Virology 251: 141-157.
Doran, P. M. 2000. Foreign protein production in plant tissue cultures. Curr. Opin. Biotech. 11: 199-204.
Fäcke, M., Janetzko, A., Shoeman, R. L. and Kräusslich, H-G. 1993. A large deletion in the matrix domain of the human immunodeficiency virus gag gene redirects virus particle assembly from the plasma membrane to the endoplasmic reticulum. J. Virol. 67: 49724980.
Fernändez-Fernändez, M. R., Mouriño, M., Rivera, J., Rodriguez, F., Plana-Durán, J. and García, J. A. 2001. Protection of rabbits against rabbit hemorrhagic disease virus by immunization with the VP60 protein expressed in plants with a potyvirus-based vector. Virology 280: 283-291.
Forster, M. J., Mulloy, B. and Nermut, M. V. 2000. Molecular modelling study of HIV p17gag (MA) protein shell utilising data from electron microscopy and X-ray crystallography. J. Mol. Biol. 298: 841-457.
Freed, E. O. 1998. HIV-1 Gag proteins: diverse functions in the virus life cycle. Virology 251: 1-15.
Friedman, R. S., Frankel, F. R., Xu, Z. and Lieberman, J. 2000. Induction of human immunodeficiency virus (HIV)-specific CD8 T-cell responses by Listeria monocytogenes and a hyperattenuated Listeria strain engineered to express HIV antigens. J. Virol. 74: 9987-9993.
Fuller, S. D., Wilk, T., Gowen, G. E., Kräusslich, H-G. and Vogt, V. M. 1997. Cryo-electron microscopy reveals ordered domains in the immature HIV-1 particle. Curr. Biol. 7: 729-738.
Garnier, L., Ratner, L., Rovinski, B., Cao, S. X. and Wills, J. W. 1998. Particle size determinants in the human immunodeficiency virus type 1 gag protein. J. Virol. 72: 4667-4677.
Gheysen, D., Jacobs, E., de Forests, F., Thiriart, C., Francotte, M., Thines, D. and De Wilde, M. 1989. Assembly and release of HIV-1 precursor Pr55^{Gap} virus-like particles from recombinant baculovirus-infected insect cells. Cell 59: 103-112.
Goulder, P. J. R., Brander, C., Annamalai, K., Mngqundanlso, N., Govender, U., Yang, Y., He, S., Hartmen, K. E., O'Callaghan, C. A., Ogg, G. S., Altfeld, M. A., Rosenberg, E. S., Cao, H., Kalams, S. A., Hammond, M., Bunce, M., Pelton, S. I., Burchett, S., A., McIntosh, K., Coovadia, H. M. and Walker, B. D. 2000. Differential narrow focusing of immunodominant human immunodeficiency virus gag-specific cytotoxic T-lymphocyte responses in infected african and caucasoid adults and children. J. Virol. 74: 5679-5690.
Gross, I., Hohenberg, H., Huckhagel, C. and Kräusslich, H-G. 1998. N-terminal extension of human Immunodeficiency virus capsid protein converts the in vitro assembly phenotype from tubular to spherical particles. J. Virol. 72: 4798-4810.
Hill, C. P., Worthylake, D., Bancroft, D. P., Christensen, A. M. and Sundquist, W. I. 1996. Crystal structures of the trimeric human immunodeficiency virus type 1 matrix protein: Implications for membrane association and assembly. Proc. Natl. Acad. Sci. USA 93: 3099-3104.
Jacks, T., Power, M. D., Masiarz, F. R., Luciw, P. A., Barr, P. J. and Varmus, H. E. 1988. Characterisation of ribosomal frameshifting in HIV-1 gag-pol expression. Nature 331: 280-283.
Jacobs, E., Gheysen, D., Thines, D., Francotte, M. and de Wilde, M. 1989. The HIV-1 Gag precursor Pr55^{gag} synthesized In yeast is myristoylated and targeted to the plasma membrane. Gene 79: 71-81.
Jowett, J. B. M., Hockley, D. J., Nermut, M. V. and Jones, I. M. 1992. Distinct signals in human immunodeficiency virus type 1 Pr55 necessary for RNA binding and particle formation. J. Gen. Virol. 73: 3079-3086.
Kapila, J., De Rycke, R., Van Montagu, M. and Angenon, G. 1997. An Agrobacterium-mediated transient gene expression system for intact leaves. Plant Science. 122. 101-108 Kazanji, M., Tartaglia, J., Franchini, G., de Thoisy, B., Talarmin, A., Contamin, H., Gessain, A. and de Thë, G. 2001. Immunogenicity and protective efficacy of recombinant human T-cell leukemia/lymphoma virus type 1 NYVAC and naked DNA vaccine candidates in squirrel monkeys (Saimiri sciureus). J. Virol. 75: 5939-5948.
Koo, M., Bendahmane, M., Lettieri, G. A., Paoletti, A. D., Lane, T. E., Fitchen, J. H., Buchmeier, M. J. and Beachy, R. N. 1999. Protective immunity against murine hepatitis virus (MHV) induced by Intranasal or subcutaneous administration of hybrids of tobacco mosaic virus that carries an MHV epitope. Proc. Natl. Acad. Sci. USA 96: 7774-7779.
Leung, N. J., Aidovini, A., Young, R., Jarvis, M. A., Smith, J. M., Meyer, D., Anderson, D. E., Carlos, M. P., Gardner, M. B. and Torres, J. V. 2000. The kinetics of specific Immune responses in rhesus monkeys inoculated with live recombinant BCG expressing SIV gag, pol, env, and nef proteins. Virology 268: 94-103.
Luciw, P. 1996. Human immunodeficiency viruses and their replication. In Fields Virology (Fields, B. N., Knipe, D. M., Howley, eds) pp 1881-1952, Lippincott—Raven Publishers, Philadelphia.
Mason, H. S., Ball, J. M., Shi, J-J., Jiang, X., Estes, M. K. and Amtzen, C. J. 1996. Expression of Norwalk virus capsid protein in transgenic tobacco and potato and its oral immunogenicity in mice. Proc. Natl. Acad. Sci. USA 93: 5335-5340.
McCormick, A. A., Kumagai, M. H., Hanley, K., Turpen, T. H., Hakim, I., Grill, L. K., Tuse, D., Levy, S. and Levy, R. 1999. Rapid production of specific vaccines for lymphoma by expression of the tumor-derived single-chain Fv epitopes in tobacco plants. Proc. Natl. Acad. Sci. USA 96: 703-708.
Mergener, K., Fäcke, M., Welker, R., Brinkmann, V., Gelderblom, H. R. and Kräusslich, H-G. 1992. Analysis of HIV particle formation using transient expression of subviral constructs in mammlian cells. Virology 186: 25-39.
Morikawa, Y., Hinata, S., Tomoda, H., Goto, T., Nakal, M., Aizawa, C., Tanaka, H and Omura, S. 1996. Complete Inhibition of human immunodeficiency virus gag myristoylation is necessary for inhibition of particle budding. J. Biol. Chem. 271: 2868-2873.
Montefiori, D. C., Safrit, J. T., Lydy, S. L., Barry, A. P., Bilska, M., Vo, H. T. T., Klein, M., Tartaglia, J., Robinson, H. L, and Rovinski, B. 2001. Induction of neutralizing antibodies and gag-specific cellular immune responses to an R5 primary Isolate of human immunodeficiency virus type 1 in rhesus macaques. J. Virol. 75: 5879-5890.
Morikawa, Y., Hockley, D. J., Nermut, M. V. and Jones, I. M. 2000. Roles of matrix, p2 and N-terminal myristoylation in human immunodeficiency virus type 1 gag assembly. J. Virol. 74: 16-23.
Nermut, M. V., Hockley, D. J., Jowett, J. B. M., Jones, I. M., Garreau, M. and Thomas, D. 1994. Fullerene-like organization of HIV gag-protein shell in virus-like particles produced by recombinant baculovirus. Virology 198: 288-296.
Nixon, D. F. and McMichael, A. J. 1991. Cytotoxic T cell recognition of HIV proteins and peptides. AIDS 5: 1049-1059.
Ono, A. and Freed, E. O. 1999. Binding of human immunodeficiency virus type 1 Gag to membrane: role of the matrix amino terminus. J. Virol. 73: 4136-4144.
Overton, H. A., Fuji, Y., Price, I. R. and Jones I. M. 1989. The protease and gag gene products of the human immunodeficiency virus: authentic cleavage and post-translational modification in an insect cell expression system. Virology 170: 107-116.
Paillart, J-C. and Göttlinger, H. G. 1999. Opposing effects of human immunodeficiency virus type 1 matrix mutations support a myristyl switch model of gag membrane targeting. J. Virol. 73: 2604-2612.
Qui, J-T., Song, R., Dettenhofer, M., Tian, Ch., August, T., Felber, B. K., Pavlakis, G. N. and Yu, X-F. 1999. Evaluation of novel human immunodeficiency virus type 1 gag DNA vaccines for protein expression in mammalian cells and induction of immune responses. J. Virol. 73: 9145-9152.
Qul, J-T., Liu, B., Tian, C., Pavlakis, G. N. and Yu, X-F. 2000. Enhancement of primary and secondary cellular Immune responses against human immunodeficiency virus type 1 gag by using DNA expression vectors that target gag antigen to the secretory pathway. J. Virol. 74: 5997-6005.
Revskaya, M. V. and Frankel, F. R. 2001. Systemic immunity and mucosal immunity are induced against human Immunodeficiency virus gag protein in mice by a new hyperattenuated strain of Listeria monocytogenes. J. Virol. 75: 2786-2791.
Riviere, Y., McChesney, M. B., Porrot, F., Tanneau-Salvadori, F., Sansonetti, P, Lopez, O, Pialoux, G., Feuillie, V., Mollereau, M. and Charnaret, S. 1995. Gag-specific cytotoxic responses to HIV type 1 are associated with a decreased risk of progression to AIDS-related complex or AIDS. AIDS Res. Hum. Retrovir. 11: 903-907.
Royer, M., Cerutti, M., Gray, B., Hong, S-S., Devauchelle, G. and Boulanger, P. 1991. Functional domain of HIV-1 gag polyprotein expressed in baculovirus-infected cells. Virology 184:417-422.
Royer, M., Hong, S-S., Gay, B., Cerutti, M. and Boulanger, P. 1992. Expression and extracellular release of human immunodeficiency virus type 1 Gag precursors by recombinant baculovirus-Infected cells. J. Virol. 66:3230-3235.
Sandefur, S., Smith, R. M., Varthakavi, V. and Spearman, P. 2000. Mapping and characterization of the N-terminal I domain of human immunodeficiency virus type 1 pr55. J. Virol. 74: 7238-7249.
Shehu-Xhilaga, M., Crowe, S. M. and Mak, J. 2001. Maintenance of the Gag/Gag-Pol ratio is important for human immunodeficiency virus type 1 RNA dimerazation and viral infectivity. J. Virol. 75: 1834-1841.
Shioda, T. and Shibuta, H. 1990. Production of human immunodeficiency virus (HIV)-like particles from cells infected with recombinant vaccinia viruses carrying the gag gene of HIV. Virology 175: 139-148.
Spearman, P., Horton, R., Ratner, L, and Kuli-Zade, I. 1997. Membrane binding of human immunodeficiency virus type 1 matrix protein in vivo supports a conformational myristyl switch mechanism. J. Virol. 71: 6582-6592.
Usha, R., Rohil, J. B., Spall, V. E., Shanks, M., Maule, A. J., Johnson, J: E. and Lonomossoff G. P. 1993. Expression of an animal virus antigenic site on the surface of a plant virus particle Virology 197: 366-374.
Vernon, S. K., Murthy, S., Wilhelm, J., Chanda, P. K., Kalyan, N., Lee, S-G. and Hung, P. P 1991. Ultrastructural characterisation of human immunodeficiency virus type 1 Gag-containing particles assembled in a recombinant adenovirus vector system. J. Gen. Virol. 72: 1243-1251.
von Schwedler, U. K., Stemmler, T. L., Klishko, V. Y., Li, S., Albertine, K. H., Davis, D. R. and Sundquist, W. I. 1998. Proteolytic refolding of the HIV-1 capsid protein amino-terminus facilitates viral core assembly. EMBO J. 17: 1555-1568.
Wang, C-T., Lai, H-Y. and Li, J-J. 1998. Analysis of minimal human immunodeficiency virus type 1 gag coding sequences capable of virus-like particle assembly and release. J. Virol. 72: 7950-7959.
Wiegers, K., Rutter, G., Kottler, H., Tessmer, U., Hohenberg, H. and Kräusslich, H-G. 1998. Sequential steps in human immunodeficiency virus particle maturation revealed by alterations of individual gag polyprotein cleavage sites. J. Virol. 72: 2846-2854.
Wigdorivitz, A., Carrillo, C., Dus Santos, M. J., Trono, K., Peralta, A., Gómez, M. C., Rios, R. D., Franzone, P. M., Sadir, A. M., Escribano, J. M. and Borca, M. V. 1999. Induction of a protective antibody response to foot and mouth disease virus in mice following oral or parenteral immunization with alfalfa transgenic plants expressing the viral structural protein VP1. Virology 255: 347-353.
Wilk, T., Gross, I., Gowen, B. E., Rütten, T., de Haas, F., Welker, R., Kräusslich, H-G., Boulanger, P. and Fuller, S. D. 2001. Organisation of Immature human immunodeficiency virus type 1. J. Virol. 75: 759-771.
Williamson, C., Morris, L., Maughan, M. F., Ping, L. H., Dryga, S. A., Thomas, R., Reap, E. A., Cilliers, T., van Harmelen, J., Pascual, A., Rarnjee, G., Gray, G., Johnston, R., Karim, S. A. and Swanstrom, R. (2003). Characterization and selection of HIV-1 subtype C isolates for use in vaccine development. AIDS Res. Hum. Retroviruses 19 (2), 133-144.
Yuan, X, Yu, X., Lee, T-H. and Essex, M. 1993. Mutations In the N-terminal regions of human immunodeficiency virus type 1 matrix protein block intracellular transport of the gag precursor. J. Virol, 67: 6387-6394.
Yusibov, V., Modelska, N., Steplewski, K., Agadjanyan, M., Weiner, D., Hooper, D. C. and Koprowski, H. 1997. Antigens produced in plants by infection with chimeric plant viruses immunize against rabies virus and HIV-1. Proc. Natl. Acad. Sci. USA 94: 5784-5788.
Zhang, G., Leung, C., Murdin, L, Rovinski, B. and White, K. A. 2000. In planta expression of HIV-1 p24 protein using an RNA plant virus-based expression vector. Mol Biotechnol. 14: 99-107.
Zhao, Y., Jones, I. M., Hockley, D. J., Nermut, M., V. and Roy, P. 1994. Complementation of human immunodeficiency virus (HIV-1) gag particle formation. Virology 199: 403-408.
Zhou, W., Parent, L. J., Wills, J. W. and Resh, M. D. 1994. Identification of a membrane-binding domain within the amino-terminal region of human immunodeficiency virus type 1 gag protein which interacts with acidic phospholipids. J. Virol. 68: 2556-2569.

1-16. (canceled)

17. A plant vector including a nucleotide sequence encoding an HIV Gag polypeptide, wherein the nucleotide sequence encoding the Gag polypeptide comprises a sequence having at least 90% sequence identity to SEQ ID NO: 1.

18. A plant vector including a nucleotide sequence encoding an HIV Gag polypeptide, wherein the nucleotide sequence encoding the Gag polypeptide comprises a sequence having at least 90% sequence identity to SEQ ID NO: 2.

19. A vector according to claim 17, which is a tobacco mosaic virus vector.

20. A vector according to claim 17, which is an Agrobacterium tumefaciens containing a T-derived plasmid construct.

21. A plant cell including a vector according to claim 17, wherein the nucleotide sequence is operably linked to control elements compatible with expression in the cell.

22. A cell according to claim 21, which is a N. benthamiana plant cell.

23. A method of producing an HIV-1 immunogenic protein or a related polypeptide which is assembled into a form of virus-like particles, the method comprising the steps of:
(a) introducing a plant vector system into a host plant cell, the vector or vector system including a nucleic acid sequence encoding the HIV-1 immunogenic protein or related polypeptide derived by substitution, deletion and/or insertion of one or more nucleotides, and/or extension or truncation of one or both ends thereof, the nucleic acid sequence having at least 90% identity to SEQ ID NO:1 or SEQ ID NO:2;
(b) causing expression of the nucleic acid sequence in the host cell; and
(c) recovering the resulting HIV-1 immunogenic protein virus-like particles or related polypeptide virus-like particles produced within the host cell.

24. A method according to claim 23, wherein the vector is a tobacco mosaic virus vector.

25. A method according to claim 23, wherein the vector is an Agrobacterium tumefaciens containing a T-derived plasmid construct.

26. A method according to claim 23, wherein the plant cell is a N. benthamiana plant cell.

27. An HIV-1 protein or polypeptide that is produced according to the method of claim 23, and which is assembled into a form of virus-like particles.

28. A protein or polypeptide according to claim 27, which is an HIV-1 Pr55 Gag protein.

29. A vaccine for use in treatment or prophylaxis of HIV infection in a mammal, the vaccine including virus-like particles of proteins or polypeptides as described in claim 27.

30. A vaccine according to claim 29, which induces an immunogenic response to the virus-like particles in a suitable susceptible host.

31. A vaccine according to claim 29, which includes a pharmaceutical excipient and/or adjuvant, and a therapeutically effective amount of the virus-like particles.
